(19) United States
(12) Patent Application Publication
(10) Pub. No.: US 2025/0263350 A1
(43) Pub. Date: Aug. 21, 2025

(54) SYSTEM FOR PRODUCING A HYDROCARBON PRODUCT FROM A SYNGAS

(71) Applicant: JOHNSON MATTHEY DAVY TECHNOLOGIES LIMITED, London (GB)

(72) Inventors: Amelia Lorna Solveig COOK, London (GB); Christopher Thomas MARTIN, London (GB)

(21) Appl. No.: 18/857,376

(22) PCT Filed: May 16, 2023
(86) PCT No.: PCT/GB2023/051271

§ 371 Date: Oct. 16, 2024

(30) Foreign Application Priority Data
Jun. 10, 2022 (GB) 2208492.5

(51) Int. Cl. C07C 1/12; B01J 23/755; C01B 32/40

(52) U.S. Cl. CPC C07C 1/12; B01J 23/755; C01B 32/40

(57) ABSTRACT

A system for producing a hydrocarbon product from a syngas, the system comprising: a syngas generation unit, a Fischer-Tropsch unit, a separation unit, a recirculation line, a derichment reactor, a carbon dioxide source, a hydrogen source, and a valve system configured to establish fluid communication in a first configuration or a second configuration.

### FIELD OF THE INVENTION

The invention relates to a system for producing a hydrocarbon product from a syngas, and a method of operating the system.

### BACKGROUND OF THE INVENTION

The Fischer-Tropsch process is a collection of chemical reactions that converts a mixture of carbon monoxide and hydrogen into liquid hydrocarbons. These reactions occur in the presence of metal catalysts, typically at temperatures of 150-300° C. and pressures of one to several tens of atmospheres. The Fischer-Tropsch process involves a series of chemical reactions that produce a variety of hydrocarbons, ideally having the formula (CₙH₂ₙ₊₂). The more useful reactions produce alkanes as follows:

(2n+1)H₂+n CO→CₙH₂ₙ₊₂+n H₂O

where n is typically 1-100, or higher. The formation of methane (n=1) is unwanted. Most of the alkanes produced tend to be straight-chain, suitable to be upgraded to produce middle-distillate fuels such as diesel and jet fuel. In addition to alkane formation, competing reactions give small amounts of alkenes, as well as alcohols and other oxygenated hydrocarbons. Co-produced water is a by-product, which is separated from the products of the Fischer-Tropsch reaction. The Fischer-Tropsch reaction is a highly exothermic reaction due to a standard reaction enthalpy (ΔH) of −165 kJ/mol CO combined.

WO2022/079408 describes a process for producing a gas stream comprising carbon monoxide comprising the steps of (a) feeding a gas mixture comprising carbon dioxide and hydrogen to a burner disposed in a reverse water-gas shift vessel and combusting it with a sub-stoichiometric amount of an oxygen gas stream to form a combusted gas mixture comprising carbon monoxide, carbon dioxide, hydrogen and steam, (b) passing the combusted gas mixture through a bed of reverse water-gas shift catalyst disposed within the reverse water-gas shift vessel to form a crude product gas mixture containing carbon monoxide, steam, hydrogen and carbon dioxide, (c) cooling the crude product gas mixture to below the dew point and recovering a condensate to form a dewatered product gas, (d) removing carbon dioxide from the dewatered product gas in a carbon dioxide removal unit to form the gas stream comprising carbon monoxide, and (e) combining carbon dioxide recovered by the carbon dioxide removal unit with the gas mixture comprising hydrogen and carbon dioxide fed to the reverse water-gas shift vessel. The product gas stream comprising carbon monoxide is fed to a Fischer-Tropsch hydrocarbon synthesis unit. A Fischer-Tropsch tail gas formed by the Fischer-Tropsch hydrocarbon synthesis unit is pre-reformed in a derichment reactor to convert species containing more than one carbon atom to methane. A gas mixture comprising the methane, and optionally non-condensable hydrocarbons recovered from the Fischer-Tropsch process, is fed to the reverse water-gas shift unit. As a result, the carbon efficiency of the process is improved.

In the event of a fault in the Fischer-Tropsch hydrocarbon synthesis unit, the whole plant needs to be shut down. Restarting the whole plant, including the synthesis gas generation unit, can take a long time and may result in wastage of the gas stream comprising carbon monoxide. An alternative to shutting down the entire plant is to isolate the derichment reactor, depressurise it and purge with nitrogen. However, this creates a number of problems, and nitrogen would be vented which is expensive and wasteful. In addition, isolation valves downstream of the derichment reactor would have to operate at a temperature of around 550° C., meaning that they would likely not perform well.

The present invention seeks to tackle at least some of the problems associated with the prior art or at least to provide a commercially acceptable alternative solution thereto.

### SUMMARY OF THE INVENTION

One aspect of the present disclosure is directed to a system for producing a hydrocarbon product from a syngas, the system comprising:
(i) a syngas generation unit comprising:
   a first inlet for supplying a flow of a first feed gas comprising hydrogen, and carbon dioxide into the syngas generation unit,
   one or more reaction zones downstream of and in fluid communication with the first inlet that convert the first feed gas into a carbon monoxide-enriched syngas, and
   a first outlet downstream of and in fluid communication with the one or more reaction zones for passing a flow of the carbon monoxide-enriched syngas from the syngas generation unit;
(ii) a Fischer-Tropsch unit comprising a reactor for converting a second feed gas comprising the carbon monoxide-enriched syngas and a recycle gas mixture to a liquid product mixture comprising the hydrocarbon product and water, the Fischer-Tropsch reactor comprising:
   a second inlet for supplying the flow of the second feed gas into the Fischer Tropsch reactor,
   a bed of Fischer-Tropsch catalyst downstream of and in fluid communication with the second inlet, the bed of Fischer-Tropsch catalyst for converting the second feed gas to the liquid product mixture comprising the hydrocarbon product and water,
   a second outlet downstream of and in fluid communication with the bed of Fischer-Tropsch catalyst for passing the liquid product mixture and a gas mixture comprising gaseous by-products and unreacted syngas from the Fischer-Tropsch reactor;
(iii) a separation unit downstream of and in fluid communication with the second outlet to separate the liquid product mixture and the gas mixture, said separation unit comprising a third outlet for the gas mixture and a fourth outlet for the liquid product mixture;
(iv) a recirculation line for conveying a portion of the gas mixture from the third outlet as the recycle gas mixture to the second feed gas fed to the second inlet;
(v) a derichment reactor for converting a further portion of the gas mixture from the third outlet to form a deriched methane-containing tail gas, the derichment reactor comprising:
   third inlet for supplying a third feed gas comprising the further portion of the gas mixture from the third outlet and steam into the derichment reactor, a source for supplying a flow of hydrogen into the derichment reactor via the third inlet or a fourth inlet,
   a bed of derichment catalyst downstream of and in fluid communication with the third inlet and the fourth inlet, the bed of derichment catalyst for converting the further portion of the gas mixture and steam to a deriched methane-containing tail gas, and a fifth outlet downstream of and in fluid communication with the bed of derichment catalyst, the fifth outlet for passing the deriched methane-containing tail gas or the flow of hydrogen from the derichment reactor, the fifth outlet in fluid communication with the first inlet;
(vi) a carbon dioxide source in fluid communication with the first inlet;
(vii) a hydrogen source in fluid communication with the first inlet; and
(viii) a valve system configured to establish fluid communication in a first configuration or a second configuration,
wherein:
in the first configuration:
   the first outlet of the syngas generation unit is in fluid communication with the second inlet of the Fischer-Tropsch reactor,
   the third outlet of the separation unit is in fluid communication with
   the third inlet of the derichment reactor, and
   the hydrogen source is not in fluid communication with the third inlet or the fourth inlet of the derichment reactor; and
in the second configuration:
   the first outlet of the syngas generation unit is not in fluid communication with the second inlet of the Fischer-Tropsch reactor,
   the third outlet of the separation unit is not in fluid communication with the third inlet of the derichment reactor, and
   the hydrogen source is in fluid communication with the third inlet or the fourth inlet of the derichment reactor.

Another aspect of the present disclosure is directed to a method of operating the system to produce a hydrocarbon product from a syngas, the method comprising:
operating the system in the first configuration,
monitoring the presence or absence of a malfunction of the Fischer-Tropsch unit, and switching the system to the second configuration in response to the presence of a malfunction of the Fischer-Tropsch unit.

### BRIEF DESCRIPTION OF THE DRAWINGS

FIG. 1 shows a flow diagram of an example of a system according to the present invention.

FIG. 2 shows a flow diagram of the example system of FIG. 1 in a first configuration.

FIG. 3 shows a flow diagram of the example system of FIG. 1 in a second configuration.

FIG. 4 shows a flow diagram of the example system of FIG. 1 in a first configuration including valves.

FIG. 5 shows a flow diagram of the example system of FIG. 1 in a second configuration including valves.

### DETAILED DESCRIPTION OF THE INVENTION

In a first aspect, the present disclosure is directed to a system for producing a hydrocarbon product from a syngas, the system comprising:
(i) a syngas generation unit comprising:
   a first inlet for supplying a flow of a first feed gas comprising hydrogen, and carbon dioxide into the syngas generation unit,
   one or more reaction zones downstream of and in fluid communication with the first inlet that convert the first feed gas into a carbon monoxide-enriched syngas, and
   a first outlet downstream of and in fluid communication with the one or more reaction zones for passing a flow of the carbon monoxide-enriched syngas from the syngas generation unit;
(ii) a Fischer-Tropsch unit comprising a reactor for converting a second feed gas comprising the carbon monoxide-enriched syngas and a recycle gas mixture to
   a liquid product mixture comprising the hydrocarbon product and water, the Fischer-Tropsch reactor comprising:
      a second inlet for supplying the flow of the second feed gas into the Fischer Tropsch reactor,
      a bed of Fischer-Tropsch catalyst downstream of and in fluid communication with the second inlet, the bed of Fischer-Tropsch catalyst for converting the second feed gas to the liquid product mixture comprising the hydrocarbon product and water,
      a second outlet downstream of and in fluid communication with the bed of Fischer-Tropsch catalyst for passing the liquid product mixture and a gas mixture comprising gaseous by-products and unreacted syngas from the Fischer-Tropsch reactor;
(iii) a separation unit downstream of and in fluid communication with the second outlet to separate the liquid product mixture and the gas mixture, said separation unit comprising a third outlet for the gas mixture and a fourth outlet for the liquid product mixture,
(iv) a recirculation line for conveying a portion of the gas mixture from the third outlet as the recycle gas mixture to the second feed gas fed to the second inlet;
(v) a derichment reactor for converting a further portion of the gas mixture from the third outlet to form a deriched methane-containing tail gas, the derichment reactor comprising:
   third inlet for supplying a third feed gas comprising the further portion of the gas mixture from the third outlet and steam into the derichment reactor,
   a source for supplying a flow of hydrogen into the derichment reactor via the third inlet or a fourth inlet,
   a bed of derichment catalyst downstream of and in fluid communication with the third inlet and the fourth inlet, the bed of derichment catalyst for converting the further portion of the gas mixture and steam to a deriched methane-containing tail gas, and
   a fifth outlet downstream of and in fluid communication with the bed of derichment catalyst, the fifth outlet for passing the deriched methane-containing tail gas or the flow of hydrogen from the derichment reactor, the fifth outlet in fluid communication with the first inlet;
(vi) a carbon dioxide source in fluid communication with the first inlet;
(vii) a hydrogen source in fluid communication with the first inlet; and
(viii) a valve system configured to establish fluid communication in a first configuration or a second configuration,
wherein:
in the first configuration:
   the first outlet of the syngas generation unit is in fluid communication with the second inlet of the Fischer-Tropsch reactor,
   the third outlet of the separation unit is in fluid communication with
   the third inlet of the derichment reactor, and
   the hydrogen source is not in fluid communication with the third inlet or the fourth inlet of the derichment reactor; and
in the second configuration:
   the first outlet of the syngas generation unit is not in fluid communication with the second inlet of the Fischer-Tropsch reactor,
   the third outlet of the separation unit is not in fluid communication with the third inlet of the derichment reactor, and
   the hydrogen source is in fluid communication with the third inlet or the fourth inlet of the derichment reactor.

Each aspect or embodiment as defined herein may be combined with any other aspect(s) or embodiment(s) unless clearly indicated to the contrary. In particular, any features indicated as being preferred or advantageous may be combined with any other feature indicated as being preferred or advantageous.

In the first configuration, the system is capable of producing a hydrocarbon product from a syngas. Advantageously, in the event of a fault in the Fischer-Tropsch unit, the separation unit and/or the recirculation line, these parts can be isolated from the syngas generation unit and derichment reactor by operating the valve system to change the system from the first configuration to the second configuration. Accordingly, the syngas generation unit and derichment reactor can be kept online while the fault is fixed and the Fischer-Tropsch unit and the separation unit are returned to operating conditions. Once the fault is fixed and the Fischer-Tropsch unit and the separation unit are returned to operating conditions, the system can be returned to the first configuration for producing the hydrocarbon product.

By keeping the syngas generation unit online during the second configuration, there may be a reduction in the start-up time of the system once the fault is fixed, typically a reduction of up to two days. Furthermore, since the syngas generation unit is kept warm, temperature cycling of the syngas generation unit is reduced, thereby increasing the lifetime of the syngas generation unit. By remaining online, the syngas generation unit can continue to generate syngas ready for reintroduction to the Fischer-Tropsch unit once the entire system is back online.

By keeping the derichment reactor online during the second configuration, a forward flow of gases may be maintained through the derichment reactor, which may provide cooling to the equipment. As a result, a dangerously high catalyst bed temperature may be avoided. Furthermore, introduction of hydrogen into the derichment reactor during the second configuration may prevent the derichment catalyst from wetting/oxidising due to the presence of the steam. In addition, the introduction of hydrogen may avoid the production of hazardous carbonyls (e.g. nickel carbonyl) on the catalyst bed. Such hazardous carbonyls may occur if the derichment reactor is isolated and not purged while the derichment reactor cools (e.g. to below 200° C.) via heat loss to surroundings with syngas on the catalyst (e.g. a nickel catalyst).

By keeping both the syngas generation unit and derichment reactor online during the second configuration, there is no need to purge the entire system in the event of a fault in the Fischer-Tropsch unit, the separation unit and/or the recirculation line. As a result, fewer reactants may be purged, meaning that fewer reactants are lost as a result of the fault.

The term “hydrocarbon product” as used herein may encompass species formed of carbon and hydrogen. The hydrocarbon product typically comprises alkanes, and typically comprise from 5 to 100, or higher, carbon atoms per molecule.

The term “syngas” or “synthesis gas” as used herein may encompass a gas mixture containing hydrogen (i.e. molecular hydrogen H₂). and carbon monoxide (i.e. CO). The syngas may comprise other species such as, for example, carbon dioxide, water and methane.

The system comprises a syngas generation unit. Syngas generation units are known in the art.

The syngas generation unit comprises one or more reaction zones downstream of and in fluid communication with the first inlet that convert the first feed gas into a carbon monoxide-enriched syngas. The one or more reaction zones may each convert the first feed gas into a carbon monoxide-enriched syngas. Alternatively, there may be a first reaction zone that forms an intermediate gas and a second reaction zone that converts the intermediate gas to the carbon monoxide-enriched syngas. This conversion includes the reverse water-gas shift conversion of carbon dioxide and hydrogen into carbon monoxide and water. Preferably at least one of the one or more reaction zones of the syngas generation unit comprise a reverse water-gas shift catalyst. The reverse water-gas shift reaction is endothermic and so heat is provided to the one or more reaction zones. The one or more reaction zones may be heated by combustion of a fuel, by heat exchange with a suitable high temperature heat exchange medium, by electrical resistance heating or by electrical induction heating.

In some arrangements, the syngas generation unit may comprise a first reaction zone downstream of and in fluid communication with the first inlet, the first reaction zone in fluid communication with an oxygen gas source, the first reaction zone comprising a burner for partially combusting the first feed gas with the oxygen gas to form a partially combusted gas mixture, and
a second reaction zone downstream of and in fluid communication with the first reaction zone, the second reaction zone comprising a bed of reverse water-gas shift catalyst for converting the partially combusted gas mixture to a carbon monoxide-enriched syngas. In such arrangements, the first reaction zone in fluid communication with an oxygen gas source and a burner. The reaction zone and burner are typically contained in a vessel. The oxygen gas source, such as a tank of oxygen gas, oxygen produced by electrolysis of water, oxygen produced by an air separation unit, or oxygen produced by a pressure or vacuum swing adsorption unit, is typically in fluid communication with the first reaction zone via an oxygen gas inlet. Any suitable source of oxygen gas may be used. The oxygen gas purity may be at least 85% by volume or 94% by volume, preferably at least 98% by volume or 99% by volume to minimise inerts such as nitrogen. The oxygen gas source is desirably provided at a pressure above that of the first feed gas fed to the burner, for example up to 8 bar above that of the first feed gas fed to the burner, because this may generate a differential velocity and promote mixing in the burner flame. The oxygen gas source may be pre-heated if desired to improve combustion. The burner is for partially combusting the first feed gas with the oxygen gas to form a partially combusted gas mixture. Typically, the burner causes oxygen gas to react with some (but not all) of the hydrogen gas in the first feed gas to form water. In other words, the amount of oxygen gas fed to the burner is sub-stoichiometric, i.e. the amount is insufficient to combust all of the hydrogen. The molar ratio of oxygen to hydrogen (O₂:H₂) is therefore typically less than 0.5:1 and may be in the range 0.02 to 0.2:1 or 0.05 to 0.15:1. The partially combusted gas mixture therefore typically comprises hydrogen, carbon dioxide and water. Combusting hydrogen may generate heat for the subsequent reverse water-gas shift reaction. Accordingly, hydrogen should preferably be provided in excess of the carbon dioxide so that sufficient hydrogen remains after combustion to drive the reaction forward over the reverse water-gas shift catalyst.

Any burner design may be used, such as burners used in autothermal or secondary steam reformers. The streams may be fed at a single point or at multiple points. Burner designs where the gas mixture is fed to a neck region of the syngas generation unit and the oxygen is fed to a central conduit passing though the neck region and opening into a combustion zone are preferred. Combustion generates a flame in a combustion zone upstream of the water-gas shift catalyst. The localized conditions in the combustion section, especially in the flame front region, may be controlled by managing the momentum of the oxygen and gas streams. The water-gas shift vessel may be orientated such that the combustion zone is above the bed of reverse water-gas shift catalyst. Such arrangements are used in autothermal or secondary steam reforming vessels and may be used in the present process, which may be termed autothermal reverse water-gas shift (ARWGS). Other arrangements of the burner and catalyst may however be used. The first feed gas is heated by the combustion to a temperature typically in the range of from 800 to 1300° C.

As discussed in more detail below, prior to entering the first inlet, the first feed gas may be pre-heated, preferably to a temperature of from 400 to 1000° C. The system may further comprise a heater to carry out the pre-heating. Alternatively, or in addition, the system may comprise a heat exchanger arranged to transfer heat from the carbon monoxide-enriched syngas to the first feed gas. Prior to entering the first inlet, the first feed gas may be pre-pressurised, preferably to a pressure of from 10 to 50 bara, more preferably to a pressure of from 20 to 30 bara. The system may further comprise means for carrying out the pre-pressurising, for example a compressor.

The syngas generation unit comprises one or more reaction zones. At least one of the one or more reaction zones of the syngas generation unit may comprise a reverse water-gas shift catalyst. Reverse water-gas shift catalysts are known in the art. The reverse water-gas shift catalyst is suitable for converting the partially combusted gas mixture to a carbon monoxide-enriched syngas. For example, carbon dioxide and hydrogen in the partially combusted gas mixture may be converted to carbon monoxide and water over the reverse water-gas shift catalyst, and any methane present in the partially combusted gas mixture may react with water to produce carbon monoxide, carbon dioxide and hydrogen. The reaction zone containing the reverse water-gas shift catalyst is typically contained in a vessel and may be contained in the same vessel as the first reaction zone, with the first inlet and first outlet formed in the walls of the vessel.

The system comprises a Fischer-Tropsch unit. Fischer-Tropsch units are known in the art. The Fischer-Tropsch unit comprises a reactor for converting a second feed gas comprising the carbon monoxide-enriched syngas and a recycle gas mixture to a liquid product mixture comprising the hydrocarbon product and water. The bed of Fischer-Tropsch catalyst is typically contained within a vessel, for example as a fixed bed. Catalysts comprising cobalt are preferred, for example catalysts comprising 5-25% wt cobalt on a porous alumina, titania or silica support may be used. The reaction is exothermic and so the catalyst is typically cooled by direct or indirect heat exchange. Fixed catalyst beds where the catalyst is disposed within reaction tubes cooled by boiling water under pressure are preferred. In a particularly preferred arrangement, the catalyst is disposed within a plurality of catalyst carriers disposed within the reaction tubes cooled by boiling water under pressure. Such carriers are described for example in WO2016/050520 A1. The Fischer-Tropsch unit may include a downstream upgrading unit in which the hydrocarbon product from the Fischer-Tropsch reactor is converted, typically by hydroprocessing using hydrogen gas, to liquid hydrocarbon products, such as kerosene, diesel and naphtha with by-product liquid petroleum gas (LPG) and non-condensable off-gases.

The second inlet and second outlet are typically formed in the walls of the vessel.

The system comprises a separation unit downstream of and in fluid communication with the second outlet to separate the liquid product mixture and the gas mixture. Such separation units are known in the art. The separation unit may comprise a conventional gas-liquid separator for separating the liquid product mixture and the gas mixture

The system comprises a derichment reactor comprising a bed of derichment catalyst. Derichment reactors and derichment catalysts are known in the art. In the derichment reactor, the bed of derichment catalyst is for converting the further portion of the gas mixture and steam into a deriched methane-containing tail gas. A derichment reactor contains a bed of derichment catalyst, such as a catalyst used for adiabatic pre-reforming.

The system further comprises a carbon dioxide source in fluid communication with the first inlet.

The system further comprises a hydrogen source. There may be two hydrogen sources. The hydrogen source in fluid communication with the first inlet may be the same or different to the hydrogen source in fluid communication with the third or fourth inlets. The hydrogen source in fluid communication with the third or fourth inlets typically comprises only very low levels of carbon, more typically substantially no carbon. The presence of high levels of carbon in this hydrogen source may result in the formation of carbonyls in the derichment reactor when the system is in the second configuration. The hydrogen source in fluid communication with the first inlet may also have a high purity, but this is not essential and lower purity hydrogen sources may be used, if desired.

The system further comprises a valve system configured to establish fluid communication in a first configuration or a second configuration. Suitable valves are known in the art, and the skilled person is capable of arranging such valves so as to establish fluid communication in the first and second configurations. Fluid communication within the system is typically established via, for example, pipes or ducts.

The system preferably further comprises:
(ix) a controller for controlling the valve system.

The controller make take the form of, for example, a pre-programmed computer. The controller may enable easy switching between the first and second configurations.

The system preferably further comprises:
(x) a carbon monoxide-enriched syngas flare unit and/or a carbon monoxide-enriched syngas storage unit, wherein: in the first configuration the first outlet of the syngas generation unit is not in fluid communication with the carbon monoxide-enriched syngas flare unit and/or the carbon monoxide-enriched syngas storage unit, and in the second configuration, the first outlet of the syngas generation unit is in fluid communication with the carbon monoxide-enriched syngas flare unit and/or the carbon monoxide-enriched syngas storage unit.

In the second configuration where the Fischer-Tropsch unit is offline, carbon monoxide-enriched syngas will still be produced by the syngas generation unit. The carbon monoxide-enriched syngas flare unit may enable disposal of such carbon monoxide-enriched syngas. The carbon monoxide-enriched syngas storage unit may enable storage of the carbon monoxide-enriched syngas for use when the Fischer-Tropsch unit is back online and the system is switched to the second configuration.

The system preferably further comprises:
(xi) means for detecting a malfunction of the Fischer-Tropsch unit.

Such means may comprise, for example, one or more temperature sensors. Detection of a malfunction may indicate to an operator that the system should be switched from the first configuration to the second configuration. The switching could be automated by the plant control system or could be manual.

The system is preferably configured to switch from the first configuration to the second configuration on detection of a malfunction of the Fischer-Tropsch unit. This may enable the system to be switched more quickly on detection of a malfunction. Accordingly, any adverse effect to the derichment reactor caused by the malfunction of the Fischer-Tropsch unit may be reduced.

The system is preferably configured to switch from the second configuration back to the first configuration once such malfunction has been rectified. This may reduce the time required to re-start the system.

Where present in the one or more reaction zones, the reverse water-gas shift catalyst may be any suitable transition metal oxide catalyst. The reverse water-gas shift catalyst preferably comprises nickel, more preferably the reverse water-gas shift catalyst comprises from 3 to 20 wt. % nickel, expressed as NiO, on a refractory metal oxide support, based on the total weight of the reverse water-gas shift catalyst. Such catalysts may be particularly suitable for carrying out a reverse-water-gas-shift reaction at favourable temperatures and pressures and at high conversion rates. In addition, such catalysts may be capable of steam reforming any hydrocarbons contained in the first feed gas.

The reverse water-gas shift catalyst may be on a suitable refractory metal oxide support. The refractory metal oxide support may comprise zirconia, alumina, calcium aluminate, magnesium aluminate, titania magnesia, or mixtures thereof. More preferably, the catalyst comprises nickel oxide on zirconia, nickel oxide on alpha-alumina, nickel oxide on calcium aluminate or nickel oxide on magnesium aluminate.

The reverse water-gas shift catalyst may be particulate, for example in the form of shaped units such as pellets, rings or extrudates, which may be lobed or fluted. The catalytically active metal, e.g. nickel, may be dispersed throughout the particulate catalyst or present only within an eggshell layer of thickness 200 to 1000 micrometres on the surface of the refractory support. Alternatively, catalyst may comprise one or more monolithic supports such as a metal or ceramic foam or honeycomb supporting the catalytically active metal. Preferably, the catalyst is a particulate catalyst, more preferably 4-hole cylinder, particularly one that is a lobed or fluted to provide a higher geometric surface area (GSA) than a similarly sized solid cylinder without increasing pressure drop. Catalysts having a GSA in the range 400-550 m² per cubic metre are preferred.

If desired, a layer of zirconia balls, pellets or tiles may be placed on top of the catalyst to protect the surface of the catalyst from irregularities in the combusting gas flow. A benefit of providing this layer is to prevent disturbance of the surface of the catalyst bed.

The exit temperature may be in the range of from 700° C. to 1050° C., preferably of from 750 to 950° C.

The Fischer-Tropsch catalyst preferably comprises cobalt, iron and/or ruthenium, more preferably cobalt. Such a catalyst may be particularly effective at catalysing Fischer-Tropsch reactions and/or enable the reaction to proceed at favourably low temperatures and/or with high yield.

The Fischer-Tropsch catalyst may be particulate, for example in the form of shaped units such as pellets, rings or extrudates, which may be lobed or fluted.

The derichment catalyst preferably comprises nickel, more preferably having a nickel content, expressed as NiO, in the range of from 30 to 90% by weight. Such catalysts may be particularly suitable for carrying out a derichment reaction at favourable temperatures and pressures and at high conversion rates.

The derichment catalyst may be particulate, for example in the form of shaped units such as pellets, rings or extrudates, which may be lobed or fluted.

Where an oxygen source is used, the system may further comprise an electrolysis unit and/or an air separation unit for providing the oxygen source. Preferably, the electricity for the electrolysis unit is generated using renewable energy.

Preferably, the system further comprises an electrolysis unit, a high purity hydrogen separation unit and/or a hydrogen storage unit for providing the hydrogen source.

Preferably, the system further comprises a hydrocarbon-water separator downstream and in fluid communication with the fourth outlet of the separation unit for separating the liquid product mixture into a hydrocarbon product stream and a water stream. The water stream may be converted to steam and directed to the third inlet of the derichment reactor.

In a further aspect, the present disclosure is directed to a method of operating the system described herein to produce a hydrocarbon product from a syngas, the method comprising:
operating the system in the first configuration,
monitoring the presence or absence of a malfunction of the Fischer-Tropsch unit, and
switching the system to the second configuration in response to the presence of a malfunction of the Fischer-Tropsch unit.

For the avoidance of doubt, the advantages and preferable features of the first aspect apply equally to this aspect.

The method preferably further comprises:
after switching the system to the second configuration, continuing to monitor the presence or absence of a malfunction of the Fischer-Tropsch unit, and
returning the system to the first configuration in response to the absence of a malfunction of the Fischer-Tropsch unit.

The first feed gas preferably has a hydrogen to carbon dioxide molar ratio of from 2:1 to 10:1. As discussed above, hydrogen should be provided in excess of the carbon dioxide so that sufficient hydrogen remains after combustion to drive the reaction forward over the reverse water-gas shift catalyst. Excess hydrogen is also desirable in view of the potential end use of the carbon monoxide-containing gas in the Fischer-Tropsch synthesis of hydrocarbons where the H₂:CO ratio is desirably about 2:1. The molar ratio of hydrogen to carbon dioxide in the first feed gas may be in the range of 1:1 to 5:1. The ratio may vary depending on the conversion of the carbon dioxide achieved over the reverse water-gas shift bed and the desired hydrogen to carbon monoxide ratio for the downstream process.

The first feed gas preferably comprises from 15 to 50% by volume carbon dioxide, preferably from 25 to 40% by volume carbon dioxide. The first feed gas fed to the syngas generation unit preferably comprises less than 10% by volume in total of other gases, such as steam, nitrogen, carbon monoxide and methane.

Any suitable source of carbon dioxide may be used. The carbon dioxide source is preferably obtained from a synthesis gas generated by partial oxidation or steam reforming of a hydrocarbon, or generated by gasification of a carbonaceous feed, or from a furnace or boiler flue gas, wherein the furnace or boiler is heated by combustion of a fossil fuel or carbonaceous wastes, or from air or seawater.

The first feed gas is preferably pre-heated to a temperature of from 400 to 1000° C., more preferably to a temperature of from 450 to 800° C., even more preferably to a temperature of from 500 to 600° C., before being passed to the first inlet of the syngas generation unit. Such pre-heating may aid combustion.

The oxygen source, where required, preferably provides oxygen at a molar ratio of oxygen to hydrogen (O₂:H₂) in the first feed gas of less than 0.5:1, preferably from 0.02 to 0.2:1, more preferably from 0.05 to 0.15:1. Such ratios may ensure partial combustion of the hydrogen gas in the first feed gas.

The carbon monoxide-enriched syngas preferably has a hydrogen to carbon monoxide molar ratio of from 1.0 to 2.5:1, preferably from 1.2 to 2.5:1, more preferably from 1.6 to 2.2:1, even more preferably from 2.0 to 2.1:1. Such ratios are close to the stoichiometric ratio of the Fischer-Tropsch reactions.

In the first configuration the temperature of the bed of Fischer-Tropsch catalyst is preferably from 150° C. to 300° C. Such a temperature may enable the Fischer-Tropsch reactions to be carried out at high efficiency and with a high yield.

The temperature of the third feed gas fed to the derichment reactor is preferably from 250 to 650° C., preferably from 300 to 550° C.

The third feed gas preferably has a steam to carbon molar ratio of from 0.2:1 to 5:1, preferably from 0.3:1 to 3:1.

The derichment reactor preferably operates at a pressure of from 10 to 50 bara.

In the second configuration, gas contacting the derichment catalyst preferably has a maximum steam to hydrogen (H₂) ratio of from 10 to 1. This may avoid damage to the derichment catalyst.

The invention includes using the system and method as described above for the derichment reactor for one or more additional parallel derichment reactors that may be fed with one or more further recycle streams suitable for conversion in the one or more additional parallel derichment reactors into methane-containing gas streams to be fed to the syngas generation unit. For example, a parallel derichment reactor may be used for converting a hydrocarbon recycle stream from a second separation unit within the Fischer-Tropsch unit to form a deriched methane-containing recycle stream, which may also be fed to the syngas generation unit. A further valve system therefore may be provided as described above to enable operation of the system in a third configuration or a fourth configuration for each of the one or more further parallel derichment reactors. Thus, the system and method may comprise a second separation unit, a further parallel derichment reactor and a further valve system configured to establish fluid communication in a third configuration or a fourth configuration,
wherein the method further comprises operating the system in the third configuration, monitoring the presence or absence of a malfunction of the Fischer-Tropsch unit, and switching the system to the fourth configuration in response to the presence of a malfunction of the Fischer-Tropsch unit, wherein:
in the third configuration:
the first outlet of the syngas generation unit is in fluid communication with the second inlet of the Fischer-Tropsch reactor,
an outlet of the second separation unit is in fluid communication with
an inlet of the further parallel derichment reactor, and
the hydrogen source is optionally not in fluid communication with the inlet of the further parallel derichment reactor; and
in the fourth configuration:
the first outlet of the syngas generation unit is not in fluid communication with the second inlet of the Fischer-Tropsch reactor,
the outlet of the second separation unit is not in fluid communication with the inlet of the further parallel derichment reactor, and
the hydrogen source is in fluid communication with the inlet of the further parallel derichment reactor.

The invention will now be described in relation to the following non-limiting example.

### Example

An example of a system according to the present invention is described with reference to FIGS. 1 to 5.

As shown in FIGS. 1 to 5, the example system (shown generally at A) is for producing a hydrocarbon product from a syngas. The system comprises a syngas generation unit 3 comprising: a first inlet 3a for supplying a flow of a first feed gas comprising hydrogen, and carbon dioxide into the syngas generation unit 3, a first reaction zone 3b downstream of and in fluid communication with the first inlet 3a, the first reaction zone 3b in fluid communication with an oxygen gas source 4, the first reaction zone 3b comprising a burner 3c for partially combusting the first feed gas with the oxygen gas to form a partially combusted gas mixture, a second reaction zone 3d downstream of and in fluid communication with the first reaction zone 3b, the second reaction zone 3d comprising a bed of reverse water-gas shift catalyst 3e for converting the partially combusted gas mixture to a carbon monoxide-enriched syngas, and a first outlet 3f downstream of and in fluid communication with the second reaction zone 3d for passing a flow of the carbon monoxide-enriched syngas from the syngas generation unit 3.

The system further comprises a Fischer-Tropsch unit 5 comprising a reactor 5a for converting a second feed gas comprising the carbon monoxide-enriched syngas and a recycle gas mixture to a liquid product mixture comprising the hydrocarbon product and water, the Fischer-Tropsch reactor 5 comprising: a second inlet 5b for supplying the flow of the second feed gas into the Fischer Tropsch reactor, a bed of Fischer-Tropsch catalyst 5c downstream of and in fluid communication with the second inlet 5b, the bed of Fischer-Tropsch catalyst 5c for converting the second feed gas to the liquid product mixture comprising the hydrocarbon product and water, a second outlet 5d downstream of and in fluid communication with the bed of Fischer-Tropsch catalyst 5c for passing the liquid product mixture and a gas mixture comprising gaseous by-products and unreacted syngas from the Fischer-Tropsch reactor (5).

The system further comprises a separation unit 6 downstream of and in fluid communication with the second outlet 5d to separate the liquid product mixture and the gas mixture, said separation unit 6 comprising a third outlet 6a for the gas mixture and a fourth outlet 6b for the liquid product mixture.

The system further comprises a recirculation line 7 for conveying a portion of the gas mixture from the third outlet 6a as the recycle gas mixture to the second feed gas fed to the second inlet 5b.

The system further comprises a derichment reactor 8 for converting a further portion of the gas mixture from the third outlet 6a to a deriched methane-containing tail gas, the derichment reactor 8 comprising: a third inlet 8a for supplying a third feed gas comprising the further portion of the gas mixture from the third outlet 6a and steam 9 into the derichment reactor 8, a fourth inlet 8b for supplying a flow of hydrogen into the derichment reactor, a bed of derichment catalyst 8c downstream of and in fluid communication with the third inlet 8a and the fourth inlet 8b, the bed of derichment catalyst 8c for converting the further portion of the gas mixture and steam to a deriched methane-containing tail gas, and a fifth outlet (8d) downstream of and in fluid communication with the bed of derichment catalyst 8c, the fifth outlet 8d for passing the deriched methane-containing tail gas or the flow of hydrogen from the derichment reactor, the fifth outlet 8d in fluid communication with the first inlet 3a.

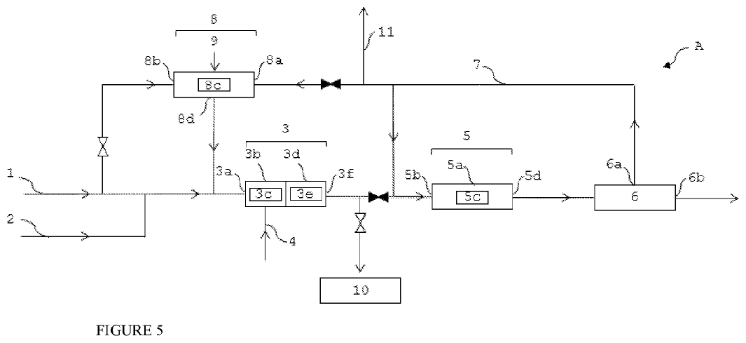

The system further comprises a carbon dioxide source 2 in fluid communication with the first inlet 3a, a hydrogen source 1 in fluid communication with the first inlet 3a, and a valve system (see FIGS. 4 and 5) configured to establish fluid communication in a first configuration or a second configuration.

The system further comprises a carbon monoxide-enriched syngas flare unit and/or a carbon monoxide-enriched syngas storage unit 10.

A small loop purge 11 is provided to prevent the unwanted build-up of inert gases, such as nitrogen, in the Fischer-Tropsch reaction loop.

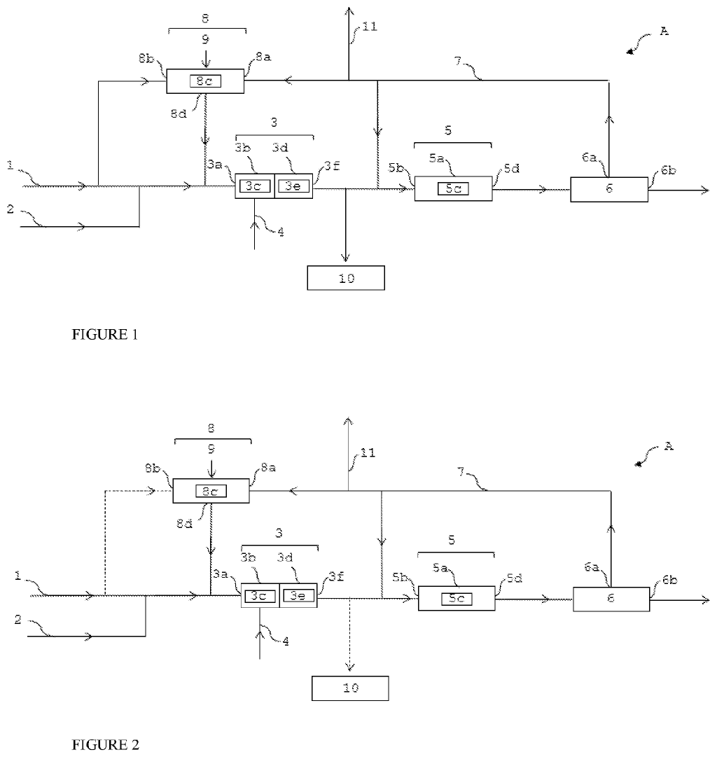

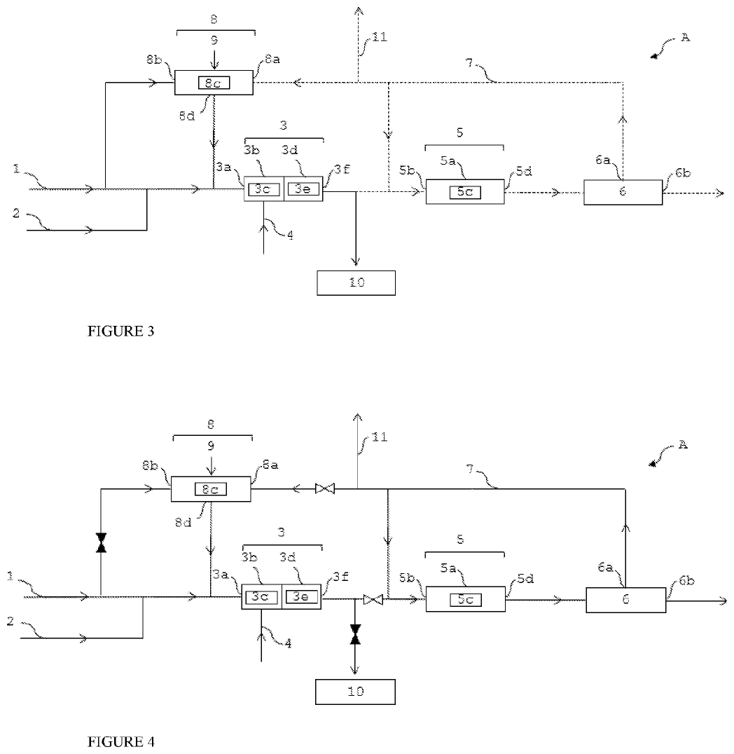

FIGS. 2 and 3 show the system in the first and second configurations, respectively, with dashes showing lines that are not in use. As can be seen from FIG. 2, in the first configuration the first outlet 3f of the syngas generation unit 3 is in fluid communication with the second inlet 5b of the Fischer-Tropsch reactor 5, the third outlet 6a of the separation unit 6 is in fluid communication with the third inlet 8a of the derichment reactor 8, the hydrogen source 1 is not in fluid communication with the fourth inlet 8b of the derichment reactor 8, and the first outlet 3f of the syngas generation unit 3 is not in fluid communication with the carbon monoxide-enriched syngas flare unit and/or the carbon monoxide-enriched syngas storage unit 10.

As can be seen from FIG. 3, in the second configuration: the first outlet 3f of the syngas generation unit 3 is not in fluid communication with the second inlet 5b of the Fischer-Tropsch reactor 5, the third outlet 6a of the separation unit 6 is not in fluid communication with the third inlet 8a of the derichment reactor 8, the hydrogen source 1 is in fluid communication with the fourth inlet 8b of the derichment reactor 8, and the first outlet 3f of the syngas generation unit 3 is in fluid communication with the carbon monoxide-enriched syngas flare unit and/or the carbon monoxide-enriched syngas storage unit 10.

FIGS. 4 and 5 show the system in the first and second configurations, respectively, and show the valve system. Unshaded vales are open and shaded valves are closed.

The foregoing detailed description has been provided by way of explanation and illustration and is not intended to limit the scope of the appended claims. Many variations in the presently preferred embodiments illustrated herein will be apparent to one of ordinary skill in the art and remain within the scope of the appended claims and their equivalents.

1. A system for producing a hydrocarbon product from a syngas, the system comprising:
(i) a syngas generation unit comprising:
a first inlet for supplying a flow of a first feed gas comprising hydrogen, and carbon dioxide into the syngas generation unit,
one or more reaction zones downstream of and in fluid communication with the first inlet that convert the first feed gas into a carbon monoxide-enriched syngas, and
a first outlet downstream of and in fluid communication with the one or more reaction zones for passing a flow of the carbon monoxide-enriched syngas from the syngas generation unit;
(ii) a Fischer-Tropsch unit comprising a reactor for converting a second feed gas comprising the carbon monoxide-enriched syngas and a recycle gas mixture to a liquid product mixture comprising the hydrocarbon product and water, the Fischer-Tropsch reactor comprising:
a second inlet for supplying the flow of the second feed gas into the Fischer Tropsch reactor,
a bed of Fischer-Tropsch catalyst downstream of and in fluid communication with the second inlet, the bed of Fischer-Tropsch catalyst for converting the second feed gas to the liquid product mixture comprising the hydrocarbon product and water,
a second outlet downstream of and in fluid communication with the bed of Fischer-Tropsch catalyst for passing the liquid product mixture and a gas mixture comprising gaseous by-products and unreacted syngas from the Fischer-Tropsch reactor;
(iii) a separation unit downstream of and in fluid communication with the second outlet to separate the liquid product mixture and the gas mixture, said separation unit comprising a third outlet for the gas mixture and a fourth outlet for the liquid product mixture,
(iv) a recirculation line for conveying a portion of the gas mixture from the third outlet as the recycle gas mixture to the second feed gas fed to the second inlet;
(v) a derichment reactor for converting a further portion of the gas mixture from the third outlet to form a deriched methane-containing tail gas, the derichment reactor comprising:
third inlet for supplying a third feed gas comprising the further portion of the gas mixture from the third outlet and steam into the derichment reactor,
a source for supplying a flow of hydrogen into the derichment reactor via the third inlet or a fourth inlet,
a bed of derichment catalyst downstream of and in fluid communication with the third inlet and the fourth inlet, the bed of derichment catalyst for converting the further portion of the gas mixture and steam to a deriched methane-containing tail gas, and
a fifth outlet downstream of and in fluid communication with the bed of derichment catalyst, the fifth outlet for passing the deriched methane-containing tail gas or the flow of hydrogen from the derichment reactor, the fifth outlet in fluid communication with the first inlet;
(vi) a carbon dioxide source in fluid communication with the first inlet;
(vii) a hydrogen source in fluid communication with the first inlet; and
(viii) a valve system configured to establish fluid communication in a first configuration or a second configuration,
wherein:
in the first configuration:
the first outlet of the syngas generation unit is in fluid communication with the second inlet of the Fischer-Tropsch reactor,
the third outlet of the separation unit is in fluid communication with the third inlet of the derichment reactor, and
the hydrogen source is not in fluid communication with the third inlet or the fourth inlet of the derichment reactor; and
in the second configuration:
the first outlet of the syngas generation unit is not in fluid communication with the second inlet of the Fischer-Tropsch reactor,
the third outlet of the separation unit is not in fluid communication with the third inlet of the derichment reactor, and
the hydrogen source is in fluid communication with the third inlet or the fourth inlet of the derichment reactor.

2. The system of claim 1, further comprising:
(ix) a controller for controlling the valve system.

3. The system of claim 1, further comprising:
(x) a carbon monoxide-enriched syngas flare unit and/or a carbon monoxide-enriched syngas storage unit,
wherein:
in the first configuration the first outlet of the syngas generation unit is not in fluid communication with the carbon monoxide-enriched syngas flare unit and/or the carbon monoxide-enriched syngas storage unit, and
in the second configuration, the first outlet of the syngas generation unit is in fluid communication with the carbon monoxide-enriched syngas flare unit and/or the carbon monoxide-enriched syngas storage unit.

4. The system of claim 1 further comprising:
(xi) means for detecting a malfunction of the Fischer-Tropsch unit.

5. The system of claim 4, wherein the system is configured to switch from the first configuration to the second configuration on detection of a malfunction of the Fischer-Tropsch unit.

6. The system of claim 5, wherein the system is configured to switch from the second configuration back to the first configuration once such malfunction has been rectified.

7. The system of claim 1 wherein at least one of the one or more reaction zones of the syngas generation unit comprise a reverse water-gas shift catalyst.

8. The system of claim 1 wherein the syngas generation unit comprises a first reaction zone downstream of and in fluid communication with the first inlet, the first reaction zone in fluid communication with an oxygen gas source, the first reaction zone comprising a burner for partially combusting the first feed gas with the oxygen gas to form a partially combusted gas mixture, and
a second reaction zone downstream of and in fluid communication with the first reaction zone, the second reaction zone comprising a bed of reverse water-gas shift catalyst for converting the partially combusted gas mixture to a carbon monoxide-enriched syngas.

9. The system of claim 7, wherein the reverse water-gas shift catalyst comprises nickel.

10. The system of claim 9, wherein the reverse water-gas shift catalyst comprises from 3 to 20 wt. % nickel, expressed as NiO, on a refractory metal oxide support, based on the total weight of the reverse water-gas shift catalyst.

11. The system of claim 1, wherein the Fischer-Tropsch catalyst comprises cobalt, iron and/or ruthenium, preferably cobalt.

12. The system of claim 1 wherein the derichment catalyst comprises nickel, preferably having a nickel content, expressed as NiO, in the range of from 30 to 90% by weight.

13. The system of claim 8, further comprising an electrolysis unit and/or an air separation unit for providing the oxygen source.

14. The system of claim 1 further comprising an electrolysis unit, a high purity hydrogen separation unit and/or a hydrogen storage unit for providing the hydrogen source in fluid communication with the third or fourth inlets and optionally in fluid communication with the first inlet.

15. A method of operating the system of claim 1 to produce a hydrocarbon product from a syngas, the method comprising:
operating the system in the first configuration,
monitoring the presence or absence of a malfunction of the Fischer-Tropsch unit, and
switching the system to the second configuration in response to the presence of a malfunction of the Fischer-Tropsch unit.

16. The method according to claim 15, the method further comprising:
after switching the system to the second configuration, continuing to monitor the presence or absence of a malfunction of the Fischer-Tropsch unit, and
returning the system to the first configuration in response to the absence of a malfunction of the Fischer-Tropsch unit.

17. The method of claim 15, wherein the first feed gas has a hydrogen to carbon dioxide molar ratio of from 2:1 to 10:1.

18. The method of claim 15, wherein the first feed gas comprises from 15 to 50% by volume carbon dioxide, preferably from 25 to 40% by volume carbon dioxide.

19. The method of claim 15, wherein the carbon dioxide source is obtained from a synthesis gas generated by partial oxidation or steam reforming of a hydrocarbon or generated by gasification of a carbonaceous feed, or from a furnace or boiler flue gas, wherein the furnace or boiler is heated by combustion of a fossil fuel or carbonaceous wastes, or from air or seawater.

20. The method of claim 15, wherein the carbon monoxide-enriched syngas has a hydrogen to carbon monoxide molar ratio of from 1.0 to 2.5:1, preferably from 1.2 to 2.5:1, more preferably from 1.6 to 2.2:1, even more preferably from 2.0 to 2.1:1.

21. The method of claim 15, wherein in the first configuration the temperature of the bed of Fischer-Tropsch catalyst is from 150° C. to 300° C.

22. The method of claim 15, wherein the temperature of the third feed gas fed to the derichment reactor is from 250 to 650° C., preferably from 300 to 550° C.

23. The method of claim 15, wherein the third feed gas has a steam to carbon molar ratio of from 0.2:1 to 5:1, preferably from 0.3:1 to 3:1.

24. The method of claim 15, wherein the derichment reactor operates at a pressure of from 10 to 50 bara.

25. The method of claim 15, wherein in the second configuration, gas contacting the derichment catalyst has a maximum steam to hydrogen (H₂) ratio of from 10 to 1.

26. The method of claim 15, wherein the system comprises a second separation unit, a further parallel derichment reactor and a further valve system configured to establish fluid communication in a third configuration or a fourth configuration,
wherein the method further comprises operating the system in the third configuration,
monitoring the presence or absence of a malfunction of the Fischer-Tropsch unit, and
switching the system to the fourth configuration in response to the presence of a malfunction of the Fischer-Tropsch unit, wherein:
in the third configuration:
the first outlet of the syngas generation unit is in fluid communication with the second inlet of the Fischer-Tropsch reactor,
an outlet of the second separation unit is in fluid communication with an inlet of the further parallel derichment reactor, and
the hydrogen source is optionally not in fluid communication with the inlet of the further parallel derichment reactor; and
in the fourth configuration:
the first outlet of the syngas generation unit is not in fluid communication with the second inlet of the Fischer-Tropsch reactor,
the outlet of the second separation unit is not in fluid communication with the inlet of the further parallel derichment reactor, and
the hydrogen source is in fluid communication with the inlet of the further parallel derichment reactor.
